(19) United States
(12) Patent Application Publication
(10) Pub. No.: US 2011/0203595 A1
(43) Pub. Date: Aug. 25, 2011

(54) Cannula Security Piece

(76) Inventor: Julia Hashemieh, Los Gatos, CA (US)

(21) Appl. No.: 13/031,603

(22) Filed: Feb. 21, 2011

Related U.S. Application Data
(60) Provisional application No. 61/305,980, filed on Feb. 19, 2010.

(51) Int. Cl. A61M 16/00

(52) U.S. Cl. 128/207.18

(57) ABSTRACT

A set of annular sleeves circumferentially mounted along sections of a nasal cannula. The sleeves are protective devices that prevent skin irritation and bed sores from developing after prolonged use of a nasal cannula. Improved stability of the cannula tube is also provided in the form of a sleeve connecting strap between ear sleeves, preventing dislocation of the cannula from the patient. The sleeves comprise tubular sections with an interior and an exterior surface. The interior surface contacts the outer diameter of the cannula breathing tube while the exterior surface contacts the patient under the nostrils, along the neck, and around the ears. The sleeves increase the cross-section of the cannula tube for improved fitment, while the strap reacts pull-off load.

### CROSS REFERENCE TO RELATED APPLICATION

This application claims the benefit of U.S. Provisional Application No. 61/305,980 filed on Feb. 19, 2010, entitled “Cannula Security Piece”

### BACKGROUND OF THE INVENTION

1. FIELD OF THE INVENTION

The present invention relates to nasal cannulas, more specifically to annular sleeves attachable to a nasal cannula for prevention of skin irritation and dislocation.

2. DESCRIPTION OF THE PRIOR ART

A nasal cannula is a useful device that supplies supplemental air flow to medical patients. These patients may require assisted breathing or increased oxygen levels, including patients recovering from a surgery, elderly patients or those that are extendedly bedridden. The device comprises a hollow plastic tube that connects to an air flow generator, extends upward towards a patient and wraps around his or her ears, and finally connects into a patient's nostrils using a set of prongs that introduce the flow of air.

A cannula is typically secured by wrapping a section around the back of each ear and tightening a Y-junction in front of the neck. Tension along the tube keeps the tube secured around each ear and the prongs secured into the patient's nostrils. However, it is common for this tension to be relaxed, causing sag along the length of the tube. Motion of the patient while wearing the cannula contributes to this relaxation. All too common, the cannula may become dislodged from the back of the patient's ears, pulling the prongs away from the nostrils and reducing their effectiveness to deliver air flow to the patient.

Along with fitment, a common compliant of nasal cannulas is the constant rubbing against the user's skin, especially in those situations where a cannula is required for long periods of treatment. The friction generated between the tube and the patient's skin can produce painful skin irritation and sores. The patient's discomfort is counterproductive to treatment, often prompting the patient to remove the cannula for periods of time, which defeats its purpose.

Several patents in the art have suggested improvements in nasal cannulas for both of these common problems. Most disclose adding pads around the ears or nostrils for the purpose of preventing skin irritation. These patents include U.S. Pat. No. 4,949,733 to Sampson, 4,699,139 to Marshall, 6,026,811 to Settle, and 5,025,805 to Nutter. These patents do not address fitment concerns, nor do they discuss preventing irritation generated by a nasal cannula along a patient's neck. U.S. Pat. No. 6,505,624 to Campbell discloses a behind-the-ear cannula retention device that clamps onto a cannula tube behind a patient's ears and tautly secures a section of cannula tubing against the patient's face. This places considerable pressure along the backside of each ear, and causes the tube to press into the patient's face, further exacerbating irritation concerns. A novel solution is required, one that retains the functionality of a nasal cannula while improving its ability to stay firmly fixed to the patient, as well as one that reduces the associated level of skin irritation.

### SUMMARY OF THE INVENTION

In view of the foregoing disadvantages inherent in the known types of nasal cannula systems now present in the prior art, the present invention provides a new nasal cannula system wherein the same can be utilized for providing convenience for the user when retaining a nasal cannula on a patient while reducing associated skin irritation.

It is therefore an object of the present invention to provide a nasal cannula system that provides secure fitment of the cannula tube around the patient's ears and under his or her nostrils.

Another object of the present invention is to provide a nasal cannula system that reduces skin irritation caused by contact between the cannula tube and the patient's bare skin.

Yet another object of the present invention is to provide a nasal cannula that is both inexpensive and simple to implement in the medical field.

Other objects, features and advantages of the present invention will become apparent from the following detailed description taken in conjunction with the accompanying drawings.

### BRIEF DESCRIPTIONS OF THE DRAWINGS

FIG. 1 shows a perspective view of the nasal cannula system and its accompanying elements.

FIG. 2 shows a side view of the nasal cannula system attached to a patient in its working form.

### DETAILED DESCRIPTION OF THE INVENTION

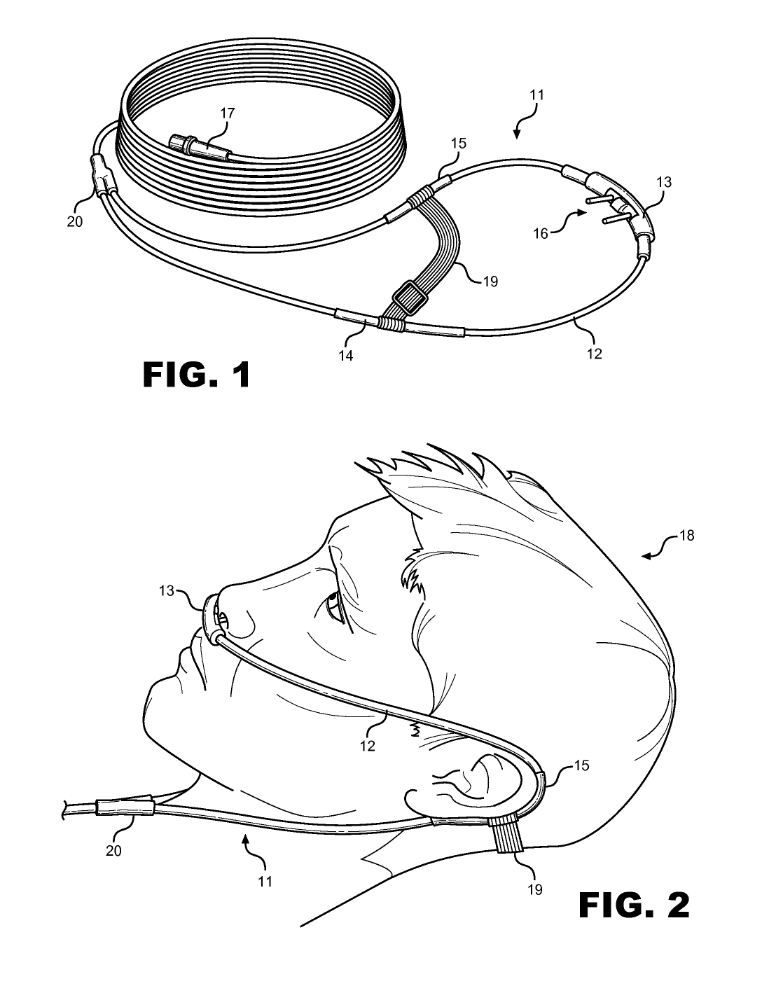

Referring now to FIG. 1, there is shown a perspective view of a nasal cannula 11 and the elements of the nasal cannula system. An annular nostril sleeve 13 covers a section of tubing 12 beneath a patient's nostrils. The nostril sleeve 13 provides openings to allow the cannula prongs 16 to protrude through. A set of annular ear sleeves 14,15 circumferentially attach to sections of tubing 12 near the patient's ears. A retaining strap 19 slideably attaches both ear sleeves 14,15 together. Finally, a neck sleeve 20 covers the junction of cannula tubing 12 along the patient's neckline.

Referring now to FIG. 2, there is shown a side view of the nasal cannula system in its working position, attached to a patient's head 18. The nostril sleeve 13 sits at the base of the nose and prevents the tubing 12 around the prongs 16 from rubbing against the patient's skin. Ear sleeves 14, 15 surround the cannula tubing 12 behind the patient's ears, increasing the cross-section of the tubing 12 in this area for more secure fitment. A retaining strap 19 wraps around the back of the patient's head 18 and ties both ear sleeves 14, 15 together. Along the patient's neck, a neck sleeve 20 attaches around the Y-junction of cannula tubing 12 to further prevent skin irritation in this region.

In use, the nasal cannula system considerably improves the comfort and security over current cannula devices. The sleeves are comprised of a soft, elastic material and covered by organic cotton or other non-allergic material to prevent skin irritation or high levels of friction. Each sleeve is a hollow, cylindrical structure with a backside cut line running longitudinally along its length. This cut line allows the sleeve to open and wrap around a section of tubing.

The retaining strap between both ear sleeves prevents movement of the patient's head from dislodging the cannula tubing. Tension along the length of the tube is maintained by the retaining strap, which is slideably mounted on each sleeve and includes an adjustment slide for accommodating different sized patients. The strap may comprise a plurality of straps or a split strap to provide improved load paths behind the patient's head if desired. Lastly, the neck sleeve provides a third region of protection from tube chaffing and skin irritation, as this region can be adjusted firmly against the patient's neck during use.

The elements of the nasal cannula strap can be used independently or together as a combination to provide maximum comfort and secure location of the cannula on a patient. The design of the sleeves and the strap are inexpensive and simple to manufacture, as well as easy to implement on an existing nasal cannulas. No modification of the cannula is necessary to add the sleeves or retaining strap, providing a cost effective solution to a common problem in the medical field.

With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1) In combination with a nasal cannula tube, a retention and comfort system that provides improved fitment and reduced skin irritation, comprising:
A set of annular sleeves with a hollow, cylindrical cross section, an interior surface and an exterior surface, said annular sleeves circumferentially mount along a sections of said cannula tube beneath a patient's nostrils, around said patient's ears and along said patient's neck, said interior surface contacts said cannula tube, and said exterior surface contacts said patient's skin;
said cannula tube beneath said patient's nostrils includes radial slots for cannula prongs to protrude.
a length-adjustable retaining strap of elastic material slideably attaches to each said annular sleeve around said patients ears

2) The apparatus of claim 1, wherein said sleeves are comprised of a soft elastic material and covered by organic cotton or other non-allergic material.

3) In combination with a nasal cannula tube, a retention and comfort system that provides improved fitment and reduced skin irritation, comprising:
A set of annular sleeves with a hollow, cylindrical cross section, an interior surface and an exterior surface, said annular sleeves circumferentially mount along a sections of said cannula tube around said patient's ears, said interior surface contacts said cannula tube, and said exterior surface contacts said patient's skin;
a length-adjustable retaining strap of elastic material slideably attaches to each said annular sleeve around said patients ears

4) The apparatus of claim 3, wherein said sleeves are comprised of a soft elastic material and covered by organic cotton or other non-allergic material.

5) In combination with a nasal cannula tube, a retention and comfort system that provides improved fitment and reduced skin irritation, comprising:
A set of annular sleeves with a hollow, cylindrical cross section, an interior surface and an exterior surface, said annular sleeves circumferentially mount along a sections of said cannula tube beneath a patient's nostrils, around said patient's ears and along said patient's neck, said interior surface contacts said cannula tube, and said exterior surface contacts said patient's skin;
said cannula tube beneath said patient's nostrils includes radial slots for cannula prongs to protrude.

6) The apparatus of claim 5, wherein said sleeves are comprised of a soft elastic material and covered by organic cotton or other non-allergic material.
